(19) United States
(12) Patent Application Publication
(10) Pub. No.: US 2008/0241061 A1
(43) Pub. Date: Oct. 2, 2008

(54) METHODS OF IMAGING EMPLOYING CHELATING AGENTS

(76) Inventors: Gregory M. LANZA, St. Louis, MO (US); Samuel A. Wickline, St. Louis, MO (US)

Correspondence Address: MORRISON & FOERSTER LLP, 12531 HIGH BLUFF DRIVE, SUITE 100, SAN DIEGO, CA, 92130-2040, US

(21) Appl. No.: 11/944,271

(22) Filed: Nov. 21, 2007

Related U.S. Application Data
(60) Provisional application No. 60/860,546, filed on Nov. 21, 2006.

(51) Int. Cl. A61K 51/12; A61M 36/00; C07F 13/00; A61P 35/00

(52) U.S. Cl. 424/1.37; 534/14

(57) ABSTRACT

Methods to image neovasculature associated with tumors using emulsions of targeted lipid/surfactant coated nanoparticles coupled to chelating agents containing radioisotopes are described.

### CROSS-REFERENCE TO RELATED APPLICATION

This application claims priority from U.S. provisional application 60/860,546 filed 21 Nov/ 2006. The contents of this document are incorporated herein by reference in their entirety.

### STATEMENT OF RIGHTS TO INVENTIONS MADE UNDER FEDERALLY SPONSORED RESEARCH

This work was supported in part by a grant from the U.S. government. The U.S. government has certain rights in this invention.

### TECHNICAL FIELD

The invention is directed to chelating agents for delivery of radioisotopes or paramagnetic ions in compositions that employ lipid/surfactant coated nanoparticles or liposomes. In particular, the invention provides chelating ligands based on nitrogen-containing ring systems that are coupled through a spacer to a lipid or hydrophobic moiety, and methods to image tumor neovasculature.

### BACKGROUND ART

Angiogenesis itself is a broadly distributed process in normal tissue growth, development, and wound healing, as well as a central feature of many pathologies, including diabetic retinopathy, and inflammatory diseases as well as cancer. The α_{σ}β₃-integrin, a heterodimeric transmembrane glycoprotein, mediates cellular adhesion to several extracellular matrix protein ligands including vitronectin, osteopontin, fibrinogen, von Willebrand factor, and denatured collagens through a specific Arg-Gly-Asp (RGD)-binding site. α_{σ}β₃-Integrin is expressed by a broad array of cell types including endothelial cells, macrophages, platelets, lymphocytes, smooth muscle cells, and tumor cells. Although it is not essential for angiogenesis, the differential upregulation of α_{σ}β₃-integrin on proliferating versus quiescent endothelial cells is frequently used as a neovascular biomarker and as an attractive target for molecular imaging and tumor anti-angiogenesis treatments.

Angiogenesis is a prominent feature of aggressive primary tumors and metastases, perhaps because tumor escape from host immune surveillance is correlated with a proliferating neovasculature and attributed to reduced endothelial expression of inflammatory markers, such as ICAM-1. Recognition of endothelial anergy has fostered further investigation of the link between tumor neovasculature and host immune responsiveness, and has motivated the search for therapeutic strategies to suppress angiogenesis and reconstitute the host immune response in combination with other immune system enhancing agents or vaccines. Specific detection of angiogenesis microanatomy, rather than the integrin itself, provides a marker correlated with aggressive tumors and diminished host immune responsiveness, which should be factored into strategic medical decisions.

Therefore, the ability to image tumor neovasculature or angiogenesis specifically is important in determining the nature of treatment.

Chelating ligands are commonly used in diagnostic and therapeutic applications to provide delivery of paramagnetic ions as contrast agents in magnetic resonance imaging or radioisotopes for imaging and therapy. The chelating agents, as complex organic molecules, can further be linked to particulate delivery systems and/or targeting moieties that bind specifically to a tissue or organ to be diagnosed or treated. Many chelating ligands are known, and a multiplicity of such ligands is described, for example, in PCT publication WO 2003/062198 which sets forth a set of very generic formulas for chelating agents in general. This publication also describes α_{σ}β₃ targeting peptidomimetics. In an illustrative embodiment, one such peptidomimetic is coupled through a spacer to a phospholipid and associated with lipid/surfactant-coated perfluorocarbon nanoparticles. More common chelating agents, including those exemplified in the above mentioned publication include ethylene diamine tetraacetic acid (EDTA); diethylene triamine pentaacetic acid (DTPA); and tetraazacyclododecane tetraacetic acid (DOTA) and their derivatives. These chelating agents have been coupled to additional moieties using bridging groups as described in U.S. Pat. Nos. 5,652,351; 5,756,605; 5,435,990; 5,358,704; 4,885,363; and several others. In addition, attachment of chelating agents through linkers to certain phospholipids has been described in PCT application PCT/US 2004/002257 and PCT application PCT/US 2005/019,966. In these applications as well, association with the phospholipid with lipid/surfactant-coated nanoparticles is described.

The specific high-resolution imaging of neovascular-rich pathology using α_{σ}β₃-paramagnetic nanoparticles has been described in many in vivo studies, however, magnetic resonance molecular imaging techniques require knowledge of pathology location for coil placement, for positioning the imaging fields-of-view, and for selection of appropriate pulse sequence and gating parameters. Therefore, the present invention envisions a high-sensitivity, low-resolution method for localizing tumor neovasculature that provides this knowledge.

The present invention is directed to a group of chelating agents particularly useful for the delivery of radioisotopes or paramagnetic metal ions to target tissues through association with lipid/surfactant-surrounded particulate carriers. Several of the chelating agents per se are known, including bis-pyridyl lysine and histidyl lysine. The compositions comprising these agents are particularly useful in diagnostic and therapeutic applications, as described below.

### DISCLOSURE OF THE INVENTION

The chelating systems of the invention are designed to be deliverable in vivo when coupled to nanoparticulate emulsions that comprise lipid/surfactant coating and are especially effective at chelating radioisotopes or paramagnetic ions when formulated in this context. As further described below, the chelating portion of the molecules of the invention is superior to alternative chelators in sequestering radioisotopes or paramagnetic ions when presented in this context. The availability of these agents permits particularly effective imaging of neovasculature associated with tumors as opposed to neovasculature associated with normal tissues and can be combined with high resolution, low sensitivity images of tumors. The radioactive, high sensitivity, low resolution formulations that contain the particulates comprising the chelating agents of the invention are relatively specific to tumor neovasculature due to the particulate nature of the delivery system. The biodistribution as mandated by the formulation itself avoids penetration into the tumor and interaction with integrin expressed on non-endothelial cells—i.e., cells not characteristic of neovasculature, and also avoids accumulation of particles in muscle where blood vessels are normal in nature. The accumulation permits identification of areas of tumor neovasculature, which can then be further imaged with a high resolution system such as SPECT-CT.

Thus, in one aspect, the invention is directed to use of an emulsion of nanoparticles targeted to α_{σ}β₃ which nanoparticles include a chelated radioisotope in a method to identify the location of angiogenesis associated with a tumor as distinct from angiogenesis in normal tissue which method comprises administering to a tumor-bearing subject an emulsion of nanoparticles targeted to α_{σ}β₃ which nanoparticles include a chelated radioisotope and obtaining a high sensitivity low resolution image of neovasculature;

optionally followed by obtaining a high-resolution, low-sensitivity image of the neovasculature in said tumor.

In another aspect, the invention is directed to modified chelating agents particularly useful in the method of the invention which are of the formula (1)

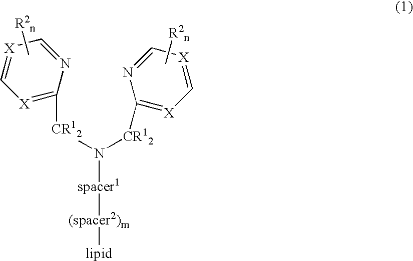

wherein

each X is independently CR¹ or N;

each R¹ is independently H or lower alkyl;

each R2 is independently halo, alkyl (1-6C), alkenyl (2-6C), or alkynyl (2-6C);

n is 0, 1 or 2;

spacer¹ is an alkylene or alkenylene chain of four or more carbons;

spacer², when present, couples spacer¹ to a lipid moiety and is a hydrophilic optionally substituted alkylene chain wherein one or more C may be replaced by N or O and wherein said chain may be substituted with one or more of OR, NR₂, ═O, COOR, CONR₂, OOCR, and/or NRCOR wherein each R is independently H or lower alkyl;

m is 0 or 1; and

lipid represents a fatty acid, a phospholipid, a sphingolipid or a steroid.

When used in the method of the invention and in other contexts, the compounds of formula (1) chelate a metal ion, in particular a radioisotope, such as ¹¹¹In or ^{99m}Tc.

In other aspects, the invention is directed to compositions comprising particulate carriers suitable for in vivo administration wherein the particulate carriers are coated with or otherwise support an outer lipid/surfactant layer which contain the compound of formula (1) embedded in such layer wherein a multiplicity of molecules of formula (1) is contained on each particle. The particles may further be coupled to a targeting ligand.

In other aspects, the invention is directed to methods to obtain magnetic resonance images, radioisotope-engendered images, and to deliver radioisotope-mediated treatments using the compositions of the invention.

### BRIEF DESCRIPTION OF THE DRAWINGS

FIGS. 1A-1D are graphs that represent tumor-to-muscle ratio of counts when radioisotopes are administered in the compositions of the invention. FIGS. 1A and 1B compare dosages of compositions containing targeted nanoparticles. FIG. 1C compares results with equivalent dosages using targeted and nontargeted nanoparticle emulsions. FIG. 1D shows competition of targeted particles containing radioisotope with targeted nanoparticles containing no radioisotope.

FIGS. 2A-2F show various tomographic CT images of rabbit hindquarters wherein the animals were or were not previously administered the compositions of the invention.

FIGS. 2A-2C show axial, sagittal and coronal reconstructions respectively from tomographic CT images of the rabbit hindquarters clearly revealing the leg, bones, and a nodular mass within the popliteal fossa wherein no invention composition was administered. The tissue within the popliteal fossa cannot be discriminated as tumor or lymph node, since relatively prominent lymph nodes are always associated with this region.

FIGS. 2D-2F show comparable images to those of 2A-2C, where, in combination with the attenuation corrected, SPECT images, the presence of neovascular signal from ^{99m}Tc α_{σ}β₃-targeted nanoparticle signal associated with a˜1 cm tissue mass located superior to the lymph node proper is readily appreciated and distinguished. Other regions of increased nuclear signal are associated with growing bone and testis, which are all are appreciated bilaterally. The pelvic signal reflects the clearance of ^{99m}Tc into the bladder. The combination of high sensitivity molecular imaging in conjunction with high resolution, CT imaging readily facilitates the discrimination of pathologic sources of neovasculature from expected sources of physiologic angiogenesis or the vasculature.

FIGS. 3A and 3B show results similar to those of FIGS. 1A-1D, but substituting ¹¹¹In for technicium.

### MODES OF CARRYING OUT THE INVENTION

The invention takes advantage of the ability of particular chelating moieties successfully to capture radioisotopes when the chelating moiety is associated with nanoparticles that have lipid/surfactant coating and which are in the size range of approximately 100-500 nanometers, preferably around 300 nanometers as an average diameter. This permits selective delivery to tumor neovasculature and permits localization of high resolution imaging of the microvasculature uniquely associated with tumors. The specificity conferred by delivery using particulate systems permits selective imaging of this neovasculature with minimal background associated with any angiogenesis in normal tissue, and with respect to other locations of the α₃β_{σ} integrin within tumor tissue not associated with the neovasculature per se. Because the nanoparticles targeted to this integrin are thus specifically associated with tumor neovasculature, a high sensitivity, low resolution image can be obtained to guide a higher resolution picture of the neovasculature.

One embodiment of the actual chelating moiety contained in the chelating agents of the invention is known in the art—bis-pyridyl lysine. However, this chelating moiety per se must be associated with nanoparticles in order to provide successful preliminary imaging.

The metal ion chelated to provide the imaging in the methods of the invention is a radioactive isotope. Particularly preferred are ¹¹¹In and ^{99m}Tc. Both of these are employed to detect and localize nascent, neovascular-rich tumors without prior knowledge.

In the present application, “angiogenesis” and “neovasculature” are sometimes used interchangeably. In each case, the integrin α_{σ}β₃ is upregulated and the targeted nanoparticles of the invention are focused on this target. Alternative targets might be employed, but this appears particularly successful.

The chelating agents of the invention containing radioisotopes are typically associated with the nanoparticles in multiples wherein a single nanoparticle will contain 4-20, preferably 6-10 chelating agents of the invention. The nanoparticles, as noted above, are also targeted to the neovasculature specifically.

The utility of ¹¹¹In α_{σ}β₃-nanoparticles in the Vx-2 rabbit tumor model has been tested along with details of its target specificity. Fluorescence and immunohistochemistry microscopy studies demonstrate that the ¹¹¹In ₃-nanoparticles were concentrated within the tumor capsule in regions rich in neovasculature and co-localized with FITC-lectin, a vascular endothelial marker. Few intratumoral α_{σ}β₃-nanoparticles were noted, and none were associated with the necrotic core, macrophages or tumor cells. This work is reported in Hu, G., et al., Int. J. Cancer (2007) 120:1951-1957.

¹¹¹In α_{σ}β₃-nanoparticles provide a high sensitivity, low-resolution signal from the tumor neovasculature that was rapidly recognized and persisted for hours. Despite the accumulation of radioactivity in reticuloendothelial clearance organs, the radiolabeled nanoparticle has potential for assessing early cancer arising in many important regions of the body including brain, head and neck, breast, and prostate. The ¹¹¹In α_{σ}β₃-nanoparticles can be used to screen for angiogenesis-rich, occult tumors or metastases in high-risk patients and guide high-resolution imaging with CT or MRI. However, ^{99m}Tc radioisotopes are preferred for their lower expense, shorter decay half-life, suitable energy γ-ray emission, and a greater radioactivity dosage safety margin.

The chelating systems of the invention are designed to be administered in pharmaceutical or veterinary compositions or in compositions employed in research protocols for diagnosis, imaging, treatment, or evaluation of possible treatment or diagnosis procedures. The chelating systems of the invention are designed to be associated with or coupled to particulate carriers contained in the compositions, typically as an emulsion.

As used herein, “particulate carriers” refers to nanoparticulates or microparticulates that perform the desired drug delivery or imaging function or generally, particles that are encapsulated by a lipid/surfactant coating or layer. The particulate carriers may, for example, be liposomes, nanoparticles, micelles, lipoproteins, or other lipid-based carriers. They may also be bubbles containing gas and/or gas precursors, particulates comprising hydrocarbons and/or halocarbons, hollow or porous particles or solids. In general, the particulate carriers may be solid particulates which may be coated with additional material, may be liquid cores surrounded by solid or liquid outer layers, or may contain gas or gas precursors again surrounded by solid or liquid outer layers. The particulate carriers may be supplied in the form of emulsions. The particulate carriers in the active compositions are coupled to targeting moieties that selectively bind to a desired tissue or location in a subject. The targeting moiety may be a ligand specific for a cognate that resides naturally on the targeted tissue or may be the cognate of an artificially supplied moiety, for example, avidin which will bind to a biotin-labeled targeted tissue.

These targeting moieties may be antibodies or fragments thereof, peptidomimetics, small molecule ligands, aptamers and the like. As noted above, they typically target α_{σ}β₃. They are coupled, either covalently or non-covalently, to the vehicles in the active composition.

Thus, the particulate carriers themselves may be of various physical states, including solid particles, solid particles coated with liquid, liquid particles coated with liquid, and gas particles coated with solid or liquid. Various carriers useful in the invention have been described in the art as well as means for coupling targeting components to those vehicles in the active composition. Such vehicles are described, for example, in U.S. Pat. Nos. 6,548,046; 6,821,506; 5,149,319; 5,542,935; 5,585,112; 5,149,319; 5,922,304; and European publication 727,225, all incorporated herein by reference with respect to the structure of the carriers. These documents are merely exemplary and not all-inclusive of the various kinds of particulate carriers that are useful in the invention.

The inert core of some embodiments can be a vegetable, animal or mineral oil, or fluorocarbon compound—perfluorinated or otherwise rendered additionally inert. Mineral oils include petroleum derived oils such as paraffin oil and the like. Vegetable oils include, for example, linseed, safflower, soybean, castor, cottonseed, palm and coconut oils. Animal oils include tallow, lard, fish oils, and the like. Many oils are triglycerides.

Fluorinated liquids are also used as cores. These include straight chain, branched chain, and cyclic hydrocarbons, preferably perfluorinated. Some satisfactorily fluorinated, preferably perfluorinated organic compounds useful in the particles of the invention themselves contain functional groups. Perfluorinated hydrocarbons are preferred. The nanoparticle core may comprise a mixture of such fluorinated materials. Typically, at least 50% fluorination is desirable in these inert supports. Preferably, the inert core has a boiling point of above 20° C., more preferably above 30° C., still more preferably above 50° C., and still more preferably above about 90° C.

Thus, the perfluoro compounds that are particularly useful in the above-described nanoparticle aspect of the invention include partially or substantially or completely fluorinated compounds. Chlorinated, brominated or iodinated forms may also be used.

With respect to any coating on the nanoparticles, a relatively inert core is provided with a lipid/surfactant coating that will serve to anchor the invention chelating systems to the nanoparticle itself. If an emulsion is to be formed, the coating typically should include a surfactant. Typically, the coating will contain lecithin type compounds which contain both polar and non-polar portions as well as additional agents such as cholesterol. Typical materials for inclusion in the coating include lipid surfactants such as natural or synthetic phospholipids, but also fatty acids, cholesterols, lysolipids, sphingomyelins, tocopherols, glucolipids, stearylamines, cardiolipins, a lipid with ether or ester linked fatty acids, polymerized lipids, and lipid conjugated polyethylene glycol. Other surfactants are commercially available.

The foregoing may be mixed with anionic and cationic surfactants.

Fluorochemical surfactants may also be used. These include perfluorinated alcohol phosphate esters and their salts; perfluorinated sulfonamide alcohol phosphate esters and their salts; perfluorinated alkyl sulfonamide alkylene quaternary ammonium salts; N,N-(carboxyl-substituted lower alkyl) perfluorinated alkyl sulfonamides; and mixtures thereof. As used with regard to such surfactants, the term “perfluorinated” means that the surfactant contains at least one perfluorinated alkyl group.

Typically, the lipids/surfactants are used in a total amount of 0.01-5% by weight of the nanoparticles, preferably 0.1-2% by weight. In one embodiment, lipid/surfactant encapsulated emulsions can be formulated with cationic lipids in the surfactant layer that facilitate the adhesion of nucleic acid material to particle surfaces. Cationic lipids include DOTMA, N-[1-(2,3-dioleoyloxy)propyl]-N,N,N-trimethylammonium chloride; DOTAP, 1,2-dioleoyloxy-3-(trimethylammonio)propane; and DOTB, 1,2-dioleoyl-3-(4′-trimethyl-ammonio)butanoyl-sn-glycerol may be used. In general the molar ratio of cationic lipid to non-cationic lipid in the lipid/surfactant monolayer may be, for example, 1:1000 to 2:1, preferably, between 2:1 to 1: 10, more preferably in the range between 1:1 to 1:2.5 and most preferably 1:1 (ratio of mole amount cationic lipid to mole amount non-cationic lipid, e.g., DPPC). A wide variety of lipids may comprise the non-cationic lipid component of the emulsion surfactant, particularly dipalmitoylphosphatidylcholine, dipalmitoylphosphatidyl-ethanol or dioleoylphosphatidylethanolamine in addition to those previously described. In lieu of cationic lipids as described above, lipids bearing cationic polymers such as polyamines, e.g., spermine or polylysine or polyarginine may also be included in the lipid surfactant and afford binding of a negatively charged therapeutic, such as genetic material or analogues there of, to the outside of the emulsion particles.

Other particulate vehicles may also be used in carrying out the method of the invention. For example, the particles may be liposomal particles, or lipoproteins such as HDL, LDL and VLDL. The literature describing various types of liposomes is vast and well known to practitioners. In general, liposomes are comprised of one or more amphiphilic moieties and a steroid, such as cholesterol. They may be unilamellar, multilamellar, and come in various sizes. These lipophilic features can be used to couple to the chelating agent in a manner similar to that described above with respect to the coating on the nanoparticles having an inert core; alternatively, covalent attachment to a component of the liposomes can be used. Micelles are composed of similar materials, and this approach to coupling desired materials, and in particular, the chelating agents applies to them as well. Solid forms of lipids may also be used.

In addition, proteins or other polymers can be used to form the particulate carrier. These materials can form an inert core to which a lipophilic coating is applied, or the chelating agent can be coupled directly to the polymeric material through techniques employed, for example, in binding affinity reagents to particulate solid supports. Thus, for example, particles formed from proteins can be coupled to tether molecules containing carboxylic acid and/or amino groups through dehydration reactions mediated, for example, by carbodiimides. Sulfur-containing proteins can be coupled through maleimide linkages to other organic molecules which contain tethers to which the chelating agent is bound. Depending on the nature of the particulate carrier, the method of coupling so that an offset is obtained between the dentate portion of the chelating agent and the surface of the particle will be apparent to the ordinarily skilled practitioner.

Further, the particles used as particulate carriers may contain bubbles of gas or precursors which form bubbles of gas when in use. In these cases, the gas is contained in a liquid or solid based coating.

In some embodiments, the particulate carriers may comprise targeting agents for alternative targets, such as fibrin clots, liver, pancreas, neurons, tumor tissue, i.e., any tissue characterized by particular cell surface or other ligand-binding moieties. In order to effect this targeting, a suitable ligand is coupled to the particle directly or indirectly. An indirect method is described in U.S. Pat. No. 5,690,907, incorporated herein by reference. In this method, the lipid/surfactant layer of a nanoparticle is biotinylated and the targeted tissue is coupled to a biotinylated form of a ligand that binds the target specifically. The biotinylated nanoparticle then reaches its target through the mediation of avidin which couples the two biotinylated components.

Alternatively, the specific ligand itself is coupled directly to the particle, preferably but not necessarily, covalently. Thus, in such “direct” coupling, a ligand which is a specific binding partner for a target contained in the desired location is itself linked to the components of the particle, as opposed to indirect coupling where a biotinylated ligand resides at the intended target. Such direct coupling can be effected through linking molecules or by direct interaction with a surface component. Homobifunctional and heterobifunctional linking molecules are commercially available, and functional groups contained on the ligand can be used to effect covalent linkage. Typical functional groups that may be present on targeting ligands include amino groups, carboxyl groups and sulfhydryl groups. In addition, crosslinking methods, such as those mediated by glutaraldehyde could be employed. For example, sulfhydryl groups can be coupled through an unsaturated portion of a linking molecule or of a surface component; amides can be formed between an amino group on the ligand and a carboxyl group contained at the surface or vice versa through treatment with dehydrating agents such as carbodiimides. A wide variety of methods for direct coupling of ligands to components of particles in general and to components such as those found in a lipid/surfactant coating in one embodiment are known in the art.

In slightly more detail, for coupling by covalently binding the targeting ligand to the components of the outer layer, various types of bonds and linking agents may be employed. Typical methods for forming such coupling include formation of amides with the use of carbodiimides, or formation of sulfide linkages through the use of unsaturated components such as maleimide. Other coupling agents include, for example, glutaraldehyde, propanedial or butanedial, 2-iminothiolane hydrochloride, bifunctional N-hydroxysuccinimide esters such as disuccinimidyl suberate, disuccinimidyl tartrate, bis[2-(succinimidooxycarbonyloxy)ethyl]-sulfone, heterobifunctional reagents such as N-(5-azido-2-nitrobenzoyloxy)succinimide, succinimidyl 4-(N-maleimidomethyl)cyclohexane-1-carboxylate, and succinimidyl 4-(p-maleimidophenyl)butyrate, homobifunctional reagents such as 1,5-difluoro-2,4-dinitrobenzene, 4,4′-difluoro-3,3′-dinitrodiphenylsulfone, 4,4′-diisothiocyano-2,2′-disulfonic acid stilbene, p-phenylenediisothiocyanate, carbonylbis(L-methionine p-nitrophenyl ester), 4,4′-dithiobisphenylazide, erythritolbiscarbonate and bifunctional imidoesters such as dimethyl adipimidate hydrochloride, dimethyl suberimidate, dimethyl 3,3′-dithiobispropionimidate hydrochloride and the like. Linkage can also be accomplished by acylation, sulfonation, reductive amination, and the like. Commercially available linking systems include the HYNIC linker technology marketed by AnorMED, Langley, BC. A multiplicity of ways to couple, covalently, a desired ligand to one or more components of the outer layer is well known in the art.

For example, methods to effect direct binding are described in detail in U.S. Pat. No. 6,676,963, incorporated herein by reference, with respect to these methods.

The foregoing discussion is not comprehensive. In a specific case which employs aptamers, it may be advantageous to couple the aptamer to the nanoparticle by the use of a cationic surfactant as a coating to the particles.

The targeting agent itself may be any ligand which is specific for an intended target site. The target site will contain a “cognate” for the targeting agent or ligand—i.e., a moiety that specifically binds to the targeting agent or ligand. Familiar cognate pairs include antigen/antibody, receptor/ligand, biotin/avidin and the like. Commonly, such a ligand may comprise an antibody or portion thereof, an aptamer designed to bind the target in question, a known ligand for a specific receptor such as an opioid receptor binding ligand, a hormone known to target a particular receptor, a peptide mimetic and the like. Certain organs are known to comprise surface molecules which bind known ligands; even if a suitable ligand is unknown, antibodies can be raised and modified using standard techniques and aptamers can be designed for such binding.

Antibodies or fragments thereof can be used as targeting agents and can be generated to virtually any target, regardless of whether the target has a known ligand to which it binds either natively or by design. Standard methods of raising antibodies, including the production of monoclonal antibodies are well known in the art and need not be repeated here. It is well known that the binding portions of the antibodies reside in the variable regions thereof, and thus fragments of antibodies which contain only variable regions, such as Fab, Fv, and scFv moieties are included within the definition of “antibodies.” Recombinant production of antibodies and these fragments which are included in the definition are also well established. If the imaging is to be conducted on human subjects, it may be preferable to humanize any antibodies which serve as targeting ligands. Techniques for such humanization are also well known.

Suitable paramagnetic metals for use in imaging include a lanthanide element of atomic numbers 58-70 or a transition metal of atomic numbers 21-29, 42 or 44, i.e., for example, scandium, titanium, vanadium, chromium, manganese, iron, cobalt, nickel, copper, molybdenum, ruthenium, cerium, praseodymium, neodymium, promethium, samarium, europium, gadolinium, terbium, dysprosium, holmium, erbium, thulium, and ytterbium, most preferably Gd(III), Mn(II), iron, europium and/or dysprosium.

For radionuclide imaging and treatment, radionuclides are included in the chelating system in a manner similar to the metal ions complexed for use in MRI described above or alternative coupling mechanisms may be used. Radionuclides may be either therapeutic or diagnostic; diagnostic imaging using such nuclides is well known and by targeting radionuclides to undesired tissue a therapeutic benefit may be realized as well. Typical diagnostic radionuclides include ^{99m}Tc, ⁹⁵Tc, ¹¹¹In, ⁶²Cu, ⁶⁴Cu, ⁶⁷Ga, and ⁶⁸Ga, and therapeutic nuclides include ¹⁸⁶Re, ¹⁸⁸Re, ¹⁵³Sm, ¹⁶⁶Ho, ¹⁷⁷Lu, ¹⁴⁹Pm, ⁹⁰Y, ²¹²Bi, ¹⁰³Pd, ¹⁰⁹Pd, ¹⁵⁹Gd, ¹⁴⁰La, ¹⁹⁸Au ¹⁹⁹Au, ¹⁶⁹Yb, ¹⁷⁵Yb, ¹⁶⁵Dy, ¹⁶⁶Dy, ⁶⁷Cu, ¹⁰⁵Rh, ¹¹¹Ag, and ¹⁹²Ir.

The nuclide can be provided to a preformed emulsion in a variety of ways. For example, ⁹⁹Tc-pertechnate may be mixed with an excess of stannous chloride and incorporated into the preformed emulsion of nanoparticles. Stannous oxinate can be substituted for stannous chloride. In addition, commercially available kits, such as the HM-PAO (exametazine) kit marketed as Ceretek® by Nycomed Amersham can be used. Means to attach various radioligands to the nanoparticles of the invention are understood in the art. As stated above, the radionuclide may not be an ancillary material, but may instead occupy the chelating agent in lieu of the paramagnetic ion when the composition is to be used solely for diagnostic or therapeutic purposes based on the radionuclide.

In addition to the chelating system of the invention, the particulate carriers may contain a therapeutic agent. These biologically active agents can be of a wide variety, including proteins, nucleic acids, pharmaceuticals, radionuclides and the like. Thus, included among suitable pharmaceuticals are antineoplastic agents, hormones, analgesics, anesthetics, neuromuscular blockers, antimicrobials or antiparasitic agents, antiviral agents, interferons, antidiabetics, antihistamines, antitussives, anticoagulants, and the like.

The chelating systems of the invention are compounds of the formula (1)

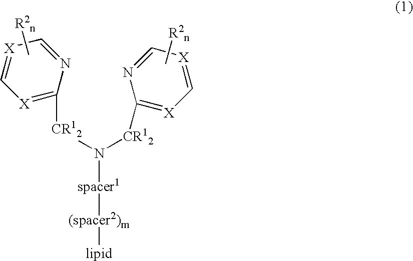

wherein

each X is independently CR¹ or N;

each R¹ is independently H or lower alkyl;

each R² is independently halo, alkyl (1-6C), alkenyl (2-6C), or alkynyl (2-6C);

n is 0, 1 or 2;

spacer¹ is an alkylene or alkenylene chain of four or more carbons;

spacer², when present, couples spacer¹ to a lipid moiety and is a hydrophilic optionally substituted alkylene chain wherein one or more C may be replaced by N or O and wherein said chain may be substituted with one or more of OR, NR₂, ═O, COOR, CONR₂, OOCR, and/or NRCOR wherein each R is independently H or lower alkyl;

m is 0 or 1; and

lipid represents a fatty acid, a phospholipid, a sphingolipid or a steroid.

In some embodiments, one or both of the nitrogen-containing rings is substituted. Such substituents are selected so as not to supply electron donor pairs to participate in the chelate. In some embodiments, one X of either or both rings is nitrogen, and the other is CR . In other embodiments, both X are nitrogen, and in still others, both X are CR¹. Preferred embodiments for R are hydrogen and methyl or ethyl in each case.

The chelating function of the molecule served by the bis-pyridyl moiety, will capture a desired positively charged metal ion. If the compositions are to be used for MRI, a paramagnetic metal will be chelated; for use in the invention method of low resolution, high sensitivity imaging, a radioisotope will be employed. Of particular interest in the method of the invention is the use of ^{99m}Tc, which is described in a review article by Liu, S., et al., Bioconjugate Chem. (1997) 8:621-636. This review describes preparation methods for various forms of this isotope (half-life 6 hours) that is particularly useful in medicine. Another embodiment often employed is ¹¹¹In which has a half-life of 2.8 days.

Spacer¹ is defined as an alkylene or alkenylene chain of four or more carbons, possibly up to six carbons or eight carbons. Spacer² may provide a cleavage site if desired and further may contain functional groups as noted above. In some embodiments, a segment of polyethylene glycol may be employed which enhances solubility in aqueous medium. Preferred functional groups contained in spacer include amides and amino groups.

Spacer2 is coupled to a hydrophobic moiety, typically a phospholipid or sphingolipid. Preferred phospholipids are those which contain functional groups for coupling to spacer², e.g. phosphatidyl ethanolamine.

In one particular embodiment of spacer¹, the alkylene chain is supplied by a lysine residue. This portion of the compounds of formula 1 can typically be synthesized as described in the art by reacting 2 moles of aldehyde-substituted pyridyl with a lysine residue that is protected at the α amino group. Subsequent reaction of the carboxyl group of the lysine residue with an alcohol or amine results in the addition of spacer². One appropriate alcohol is polyethylene glycol, typically containing 40-60 monomers, preferably 45-50 monomers. Other alcohols are amines are those of o-amino-or hydroxyl-carboxylic acids.

As noted above, a preferred embodiment of the lipid moiety is phosphatidyl ethanolamine. Any carboxyl group of the spacer² residue provides ready access to reaction with phosphatidyl ethanolamine. The acyl groups associated with the phosphatidyl ethanolamine may be of varying lengths, but should be long enough to provide a hydrophobic anchor. Typically, the acyl groups will comprise at least 12 carbon atoms and acyl groups in the range in 12-24 carbon atoms are contemplated. The acyl groups may be saturated or unsaturated but preferably are saturated.

The following preparations and examples are offered to illustrate but not to limit the invention.

### PREPARATION A

### Preparation of Targeting Agents to α_{σ}β₃

A. DSPE-PEG(2000)Maleimide-Mercaptoacetic Acid Adduct,

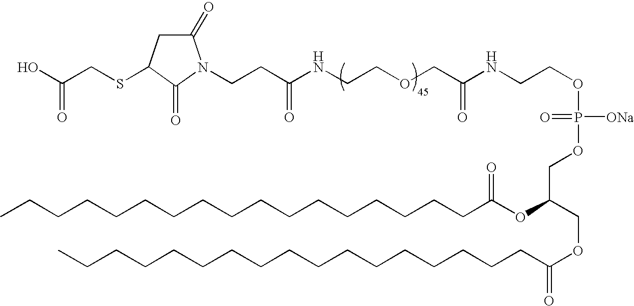

is first prepared as follows:

1,2-Distearoyl-sn-Glycero-3-Phosphoethanolamine-N-[Maleimide(Polyethylene Glycol)2000] is dissolved in DMF and degassed by sparging with nitrogen or argon. The oxygen-free solution is adjusted to pH 7-8 using DIEA, and treated with mercaptoacetic acid. Stirring is continued at ambient temperatures until analysis indicates complete consumption of starting materials. The solution is used directly in the following reaction.

The DSPE-PEG(2000)Maleimide-Mercaptoacetic Acid Adduct is then coupled to 2- [({4-[3-(N-{2-[(2R)-2-((2R)-2-amino-3-sulfopropyl)-3-sulfopropyl]ethyl }carbamoyl)-propoxy]-2,6-dimethylphenyl } sulfonyl)amino](2S)-3-({7-[(imidazol-2-ylamino)methyl]-1-methyl-4-oxo(3-hydroquinolyl)}carbonylamino)propanoic acid to obtain

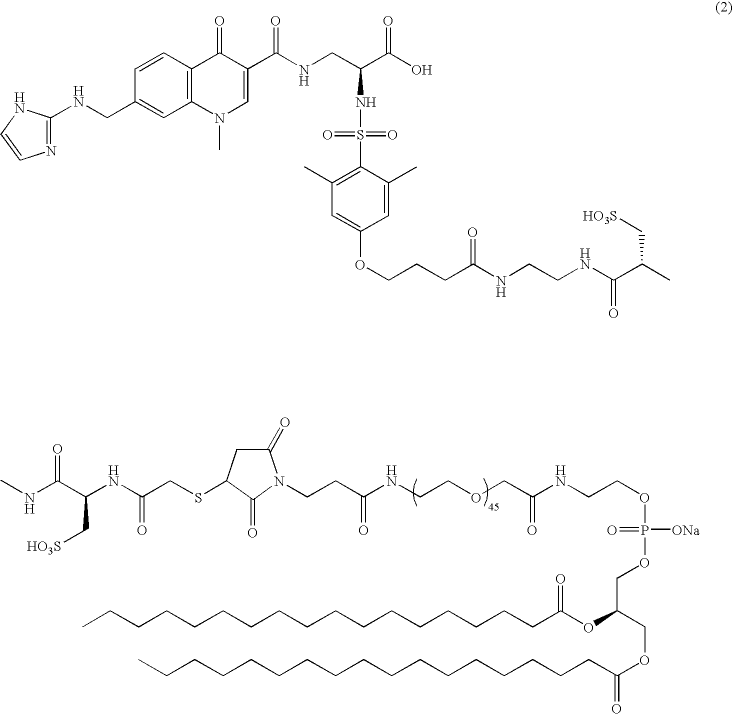

as follows:

The adduct solution above is pre-activated by the addition of HBTU and sufficient DIEA to maintain pH 8-9. To the solution is added 2-[({4-[3-(N-{2-[(2R)-2-((2R)-2-amino-3-sulfopropyl)-3-sulfopropyl]ethyl }carbamoyl)propoxy]-2,6-dimethylphenyl}sulfonyl)amino]-(2S)-3-({7-[(imidazol-2-ylamino)methyl]-1-methyl-4-oxo(3-hydroquinolyl) }carbonylamino)-propanoic acid, and the solution is stirred at room temperature under nitrogen for 18 h. DMF is removed in vacuo and the crude product is purified by preparative HPLC to obtain the conjugate.

B. Using similar procedures, a derivatized targeting agent of formula (2A) was obtained.

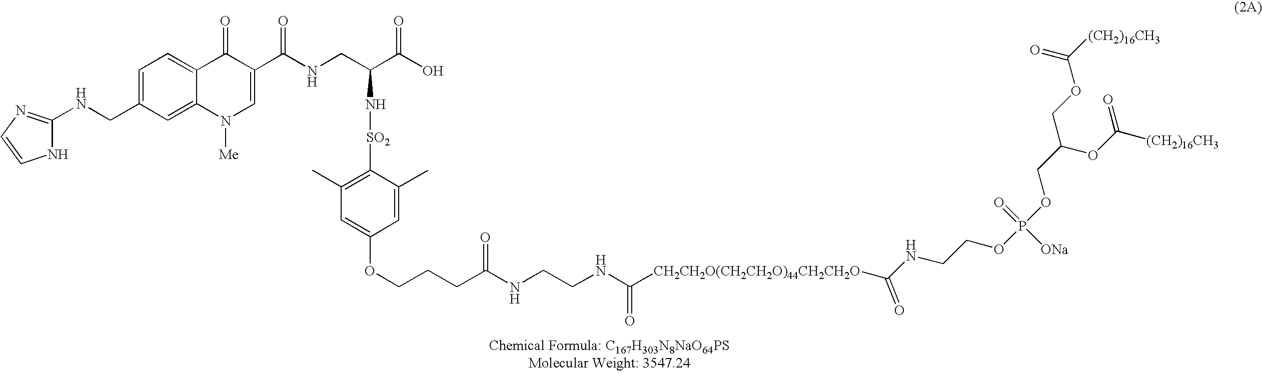

### PREPARATION B

### Preparation of Nanoparticles

A. In one embodiment, the nanoparticles are produced as described in Flacke, S., et al., Circulation (2001) 104:1280-1285. Briefly, the nanoparticulate emulsions are comprised of 40% (v/v) perfluorooctylbromide (PFOB), 2% (w/v) of a surfactant co-mixture, 1.7% (w/v) glycerin and water representing the balance.

The surfactant of control, i.e., non-targeted emulsions includes 60 mole % lecithin (Avanti Polar Lipids, Inc., Alabaster, Ala.), 8 mole % cholesterol (Sigma Chemical Co., St. Louis, Mo.) and 2 mole % dipalmitoyl-phosphatidylethanolamine (DPPE) (Avanti Polar Lipids, Inc., Alabaster, Ala.).

α_{σ}β₃-Targeted paramagnetic nanoparticles are prepared as above with a surfactant co-mixture that includes: 60 mole % lecithin, 0.05 mole % N-[{w-[4-(p-maleimidophenyl)-butanoyl]amino }poly(ethylene glycol)2000]1,2-distearoyl-sn-glycero-3-phosphoethanolamine (MPB-PEG-DSPE) covalently coupled to the α_{σ}β₃-integrin peptidomimetic antagonist (Bristol-Myers Squibb Medical Imaging, Inc., North Billerica, Mass.), 8 mole % cholesterol, 30 mole % Gd-DTPA-BOA and 1.95 mole % DPPE.

The components for each nanoparticle formulation are emulsified in a M110S Microfluidics emulsifier (Microfluidics, Newton, Mass.) at 20,000 PSI for four minutes. The completed emulsions are placed in crimp-sealed vials and blanketed with nitrogen.

Particle sizes are determined at 37° C. with a laser light scattering submicron particle size analyzer (Malvern Instruments, Malvern, Worcestershire, UK) and the concentration of nanoparticles is calculated from the nominal particle size (i.e., particle volume of a sphere). Most of the particles have diameters less than 400 nm.

Perfluorocarbon concentration is determined with gas chromatography using flame ionization detection (Model 6890, Agilent Technologies, Inc., Wilmington, Del.). One ml of perfluorocarbon emulsion combined with 10% potassium hydroxide in ethanol and 2.0 ml of internal standard (0.1% octane in Freon®) is vigorously vortexed then continuously agitated on a shaker for 30 minutes. The lower extracted layer is filtered through a silica gel column and stored at 4-6° C. until analysis. Initial column temperature is 30° C. and is ramped upward at 10° C./min to 145° C.

B. In another embodiment, the emulsified perfluorooctylbromide (PFOB) nanoparticles, prepared as reported earlier by Winter, P. M., et al., Cancer Res. (2003) 63:5838-5843; Schmieder, A., et al., Magn. Reson. Med. (2005) 53:621-627; and Hu, G., et al., Int. J. Cancer (2007) 120:1951-1957. They contained 20% (v/v) of PFOB (Exfluor Corp., Round Rock, Tex.), 2% (w/v) of a surfactant, and deionized water for the balance. The surfactant co-mixture for the integrin-targeted particles included 3-5 mole % bis-pyridyl-lysine-caproyl-phosphatidylethanolamine, 0.1 mole % vitronectin antagonist complexed to PEG2000-phosphatidylethanolamine of Formula (2), and purified egg PC (Avanti Polar Lipids, Inc.) for balance. The surfactant commixture was dissolved in chloroform, evaporated under reduced pressure, and dried in 50° C. vacuum overnight into a lipid film. The surfactant was coarse blended with perfluorooctylbromide (PFOB) and distilled, deionized water then emulsified with a Microfluidics M110S fluidizer (Microfluidics) at 20,000 psi for 4 minutes. α_{σ}β₃-targeted particles were measured with a Malvern Dynamic Light Scattering Zetasizer 4 System (Malvern Instruments, Ltd.) at 37° C. were typically 270 nm diameter with a polydispersity index of 0.2. The bioactivity of the α_{σ}β₃-targeted nanoparticles was confirmed and monitored using an in vitro vitronectin cell adhesion assay.

### PREPARATION C

Labeling α_{σ}β₃-targeting Particles With ^{99m}Tc Radioisotope (Comparative Example)

Several lipophilic chelates were synthesized and evaluated for radiolabeling perfluorocarbon nanoparticles for comparison. Briefly, these lipid-chelates included 6-hydrazinonicotinic-phosphatidylethanolamine (HYNIC-PE), diethylenetriamene pentaacetate-caproyl-phosphatidylethanolamine (DTPA-cap-PE), Gly-Gly-Gly-caproyl-phosphatidyl-ethanolamine (TriGly-cap-PE), Gly-Gly-Gly-Asp-caproyl-phosphatidyl-ethanolamine (triGly-Asp-cap-PE), N2S2-phosphatidylethanolamine (N2S2-PE), and N2S2-NH₂-phosphatidylethanolamine (N2S2-NH2-PE). Stannous tartrate reductions of ^{99m}Tc with a tricineintermediate shuttle step were used to minimize the formation of ^{99m}TcO₂ during metalation. ^{99m}Tc was coupled to the bis-pyridyl-lysine through a tricarbonyl precursor as described below.

The goal of coupling 6 to 10 ^{99m}Tc isotopes per nanoparticle with high efficiency (>90%) required the synthesis, screening and testing of several candidate lipophilic chelates. Table 1 briefly summarizes ^{99m}Tc coupling results to nanoparticles and in selected instances the free chelate when both were studied.

The best results were achieved with the tridentate bis-pyridyl-lysine-phosphatidylethanolamine conjugates of formulas (3) and (4) followed by the bidentate histidine-phosphatidylethanolamine and the lipid-modified HYNIC chelates. DTPA-PE performed poorly and the two TriGly lipophilic compounds were ineffective. The phospholipid derivatives of commonly used tetradentate N2S2 chelates bound the ^{99m}Tc in solution, but functioned poorly when incorporated into the nanoparticle lipid surfactant, despite various pH adjustments to the in-process conditions.

TABLE 1
Comparison of the ^{99m}Tc Radiolabeling Efficiency using
Different Lipophilic Chelates Incorporated into Perfluorocarbon
Nanoparticles or as the Free Lipid-Chelate

Yield achieved

| Chelators | Nanoparticle | Free |
|---|---|---|
| DTPA-cap-PE | 27% | N/A |
| TriGly-cap-PE | 0% | N/A |
| TriGly-Asp-cap-PE | 10% | N/A |
| Hynic-cap-PE | 75% | N/A |
| His-cap-PE | 70% | N/A |
| Bis-Py-Lys-cap-PE | 90% | N/A |
| Bis-Py-Lys-PEG-cap-PE | 90% | N/A |
| N2S2-PE | 0% | 93% |
| N2S2-amino-PE | 38% | 67% |

### PREPARATION D

### Preparation of ^{99m}Tc-tricarbonyl Precursor and ^{99m}Tc Nanoparticles

Sodium borohydride NaBH4 (0.53 M), sodium carbonate (0.14 M), and sodium tartrate (0.24 M) in 660 μl deionized water were admixed in a glass serum vial. The vial was purged with carbon monoxide for 20 min, then 2368 MBq of sodium pertechnetate ^{99m}TcO₄ was added, and the contents heated at 100° C. for 20 min. After equilibration to atmospheric pressure, the reaction mixture was adjusted to pH 7 with a 1:3 mixture of 0.1 M phosphate buffer (pH 7.4): 1 M HCl and purity was determined by HPLC as described below. The reaction mixture was combined with 50-100 μL nanoparticles containing 6-10 molecules of the chelating moieties of formulas (3) or (4) in water bath for 30 min at 40° C. The nanoparticle radiolabeling yield was greater than 90% as determined by TLC developed with 0.1M sodium acetate pH 5.18:methanol:water (20:100:200), which achieved approximately 6 atoms of ^{99m}Tc per nanoparticle.

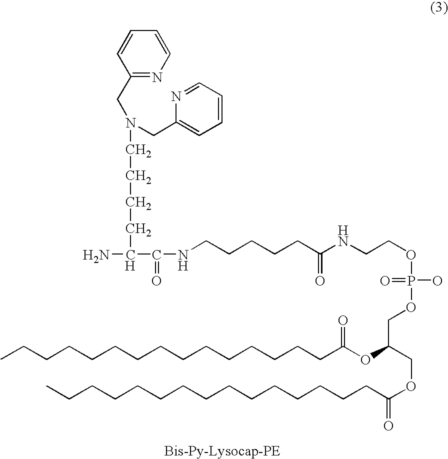

In addition to the compound of formula (3), a compound of formula (4), Bis-Py-Lyso-PEG-cap-PE was used. In this compound (PEG)₄₅ is coupled to the carboxyl of lysine and to the amino group of ω-amino caproic acid.

The formation of fac-[^{99m}Tc(OH₂)₃(CO)₃]⁺ was confirmed by reverse-phase HPLC system (Waters Corporation) and gamma counter (PerkinElmer Life And Analytical Sciences, Inc.) for detection. HPLC conditions included: Waters SymmetryShield™ RP8 3.5 μm, 4.6×250 mm, reversed-phase column and a mobile phase gradient of 0.05 M triethylammonium phosphate (TEAP) pH 2.68 and methanol (MeOH). The applied gradient was: A, 0 to 3 min 100% TEAP; 3 to 6 min, from 100% to 75% TEAP; 6 to 9 min from 75% to 66% TEAP and B, 34% to 100% MeOH from 9 to 20 min, 100% MeOH from 20 to 27 min, 100% MeOH to 100% TEAP from 27 to 30 min. The flow rate was 1 mL/min at ambient temperature.

### EXAMPLE 1

VX-2 Rabbit Tumor Model: Male New Zealand White rabbits (˜2 kg) were anesthetized with intramuscular ketamine and xylazine. Left hind leg of each animal was shaved, sterile prepped, and infiltrated with Marcaine™. A 2-3 mm³ Vx-2 carcinoma tumor (DCTD Tumor Repository, National Cancer Institute, Frederick, Mass.) was implanted at a depth of ˜0.5 cm through a small incision into the popliteal fossa. Anatomical planes were closed and secured with a single absorbable suture. The skin was sealed with Dermabond™ skin glue. Animals were recovered by reversing the effect of ketamine and xylazine with yohimbine.

Twelve to sixteen days after Vx-2 tumor implant, rabbits were anesthetized with 1-2% of Isoflurane™, intubated, and ventilated. An intravenous catheter was placed in a marginal ear vein of each rabbit for injection of the radiolabeled nanoparticles. Animals were monitored physiologically while anesthetized in accordance with a protocol approved by the Animal Studies Committee at Washington University Medical School.

Planar imaging studies: Twenty-one rabbits implanted with VX-2 tumors were randomized into 5 treatment groups to assess the tumor-to-muscle ratio (TMR) contrast response. The treatment groups (grps) selected were used to establish an optimal dosage for ^{99m}Tc α_{σ}β₃-nanoparticles (grps 1-3), to compare α_{σ}β₃-targeted versus nontargeted ^{99m}Tc nanoparticles (grps 2 vs. 4), and to demonstrate homing specificity of ^{99m}Tc α_{σ}β₃-nanoparticles competitively inhibited by unlabeled α_{σ}β₃-nanoparticles (grps 2 vs. 5).

1) 11 MBq/kg ^{99m}Tc α_{σ}β₃-nanoparticles (n=5)

2) 22 MBq/kg ^{99m}Tc α_{σ}β₃-nanoparticles (n=4)

3) 44 MBq/kg ^{99m}Tc α_{σ}β₃-nanoparticles (n=4)

4) 22 MBq/kg nontargeted ^{99m}Tc nanoparticles (n=4)

5) 22 MBq/kg ^{99m}Tc α_{σ}β₃-nanoparticles co-administered with 20-fold excess of unlabeled α_{σ}β₃-nanoparticles (n=4).

Total injection volume (0.3 ml/kg) was preserved for groups 1 to 4 with inclusion of control nanoparticles (i.e., nontargeted, unlabeled).

For planar imaging studies, rabbits were positioned 3 cm directly below a high-energy pinhole collimator (3 mm aperture) and imaged with a clinical Genesys single-head, gamma camera (Philips Medical Systems). The images were acquired for 15 minutes dynamically over 2 hours beginning 71/2 minutes after injection using a 20% window centered at 140 keV and a resolution of 128×128×16. Image stacks were exported in DICOM format to a Linux workstation and processed with ImageJ software (located on the World Wide Web at rsb.info.nih.gov/ij/). Regions-of-Interest (ROI) of comparable size were manually placed around the tumor signal, muscle, and background regions to determine average pixel activity.

^{99m}Tc signals from the tumor neovasculature dynamically acquired for the first two hours following injection of ^{99m}Tc α_{σ}β₃-nanoparticles are presented as the tumor-muscle-ratio.

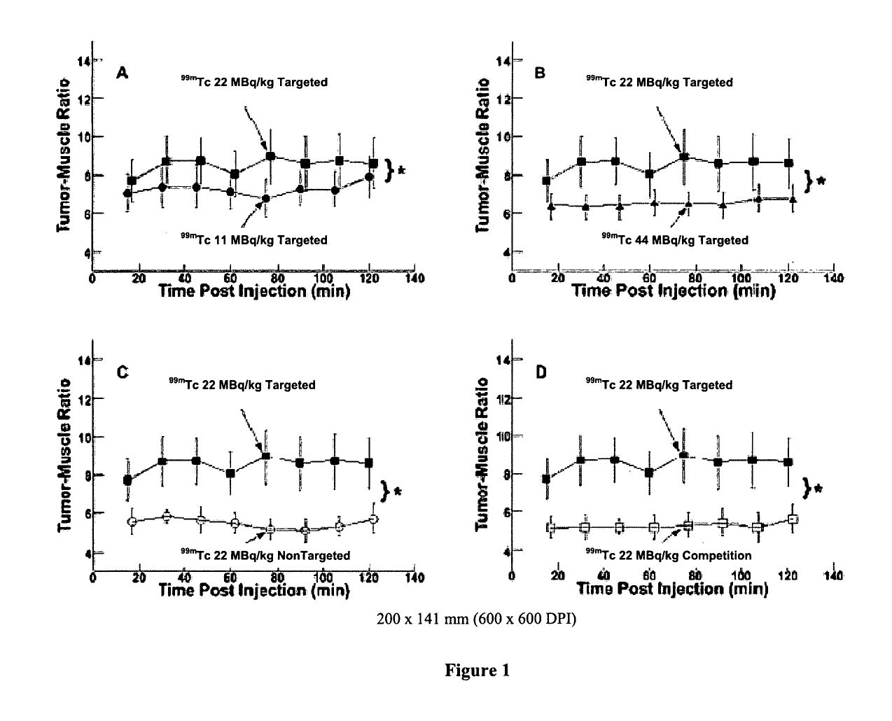

^{99m}Tc α_{σ}β₃-nanoparticles administered at 11 MBq/kg had early contrast enhancement (TMR) after 15 minutes (7.08±0.97) that was comparable to the initial signal appreciated with the 22 MBq/kg dosage (7.71±1.15) but then remained lower (p<0.05) over the remaining 2-hour study interval (11 MBq/kg, 7.32±0.12 versus 22 MBq/kg, 8.56±0.13) (FIG. 1A).

The TMR in rabbits receiving 44 MBq/kg of ^{99m}Tc α_{σ}β₃-nanoparticles was poorer (p<0.05) than the 22 MBq/kg responses at 15 minutes (6.38±0.48) and remained lower (p<0.05) over the remaining 2 hours (6.55±0.07, FIG. 1B). These results suggest that ^{99m}Tc α_{σ}β₃-nanoparticles dosed above 22 MBq/kg saturated the available α_{σ}β₃-integrin binding sites and the excess circulating activity increased the background measured in the highly vascular muscle reference.

Nontargeted ^{99m}Tc nanoparticles at the 22 MBq/kg dose had lower (p<0.05) neovascular signal (TMR) at 15 minutes post injection (5.54±0.47) than the ^{99m}Tc α_{σ}β₃-nanoparticles given at 22 MBq/kg (8.56±0.13, p<0.05) (FIG. 1C) or 11 MBq/kg (7.32±0.12). This difference persisted throughout the 2-hour study interval (p<0.05).

In vivo competitive inhibition of ^{99m}Tc α_{σ}β₃-targeted nanoparticles (22 MBq/kg) with non-labeled α_{σ}β₃-nanoparticles diminished (p<0.05) the tumor signal to a level equivalent to the nontargeted nanoparticles at 15 minutes (5.16±0.31) and over the 2-hour study (5.31±0.06, FIG. 1D).

SPECT-CT Imaging: This was illustrated using a clinical Precedence SPECT/CT 16-slice scanner (Philips Medical Systems). A male New Zealand White rabbit (˜2 kg) was anesthetized with 1-2% of Isoflurane™, intubated, and ventilated. Venous access was established in the right ear vein, and the animal was positioned prone, feet first on the table. The animal received 11 MBq/kg of ^{99m}Tc α_{σ}β₃-nanoparticles. Thirty minutes post-injection, two overlapping rectangular CT and SPECT regions were selected to register and to attenuation correct the SPECT images (FOV 350 mm, matrix 512×512, CT slice thickness 3.3 mm). The multislice CT settings were 250 mAs/slice, at 120 kV. SPECT image acquisition consisted of 64, 30-second projections (matrix 128×128 pixels) using low-energy, high-resolution collimators with a 2.19 zoom and a 27.3 cm×27.3 cm mask.

Reconstruction of the SPECT volume from tomographic projections was performed on the JETStream Workspace 2.5.1 workstation (Philips Medical Systems) with AutoSPECT Plus 3.0 software package using a 3D ordered subsets expectation maximization reconstruction algorithm, Astonish (Philips Medical Systems), which included CT attenuation map, scatter and radioisotope decay correction. Co-registration of CT and SPECT reconstructed image sets were performed using Syntegra (version 2.3.1) package on JETStream Workspace.

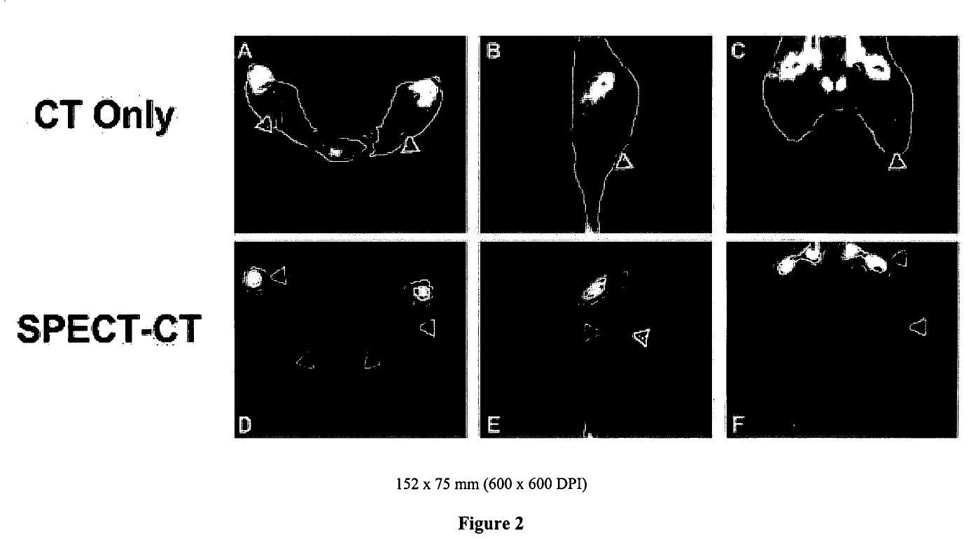

FIGS. 2A-2F present two-dimensional tomographic CT images of the rabbit hindquarters clearly revealing the leg, bones, and a nodular mass within the popliteal fossa. The soft tissue masses observed bilaterally within the popliteal fossa (FIG. 2A) cannot be discriminated as tumor or lymph node, since prominent lymph nodes are always associated with this region. In combination with the attenuation- and decay-corrected SPECT images, the presence of neovascular signal derived from ^{99m}Tc α_{σ}β₃-nanoparticles associated with a ˜1 cm tissue mass located in the superior right fossa is readily appreciated and distinguished from the adjacent lymph node. Other regions of increased nuclear signal are associated with bone and prepubertal testes. These contrast signals are appreciated bilaterally and occur in organs high in angiogenesis and blood flow. The combination of high sensitivity molecular imaging in conjunction with high-resolution CT imaging facilitated the discrimination of pathologic sources of neovasculature from expected sources of physiologic angiogenesis.

Histology: After imaging, animals were euthanized and tumors resected, weighed and quickly frozen in OCT for routine histopathology. In two animals, testes were excised as a positive control to confirm neovascularity within the spermatic cords. Acetone-fixed, frozen tissues were sectioned (5 μm) and routinely stained with hematoxylin and eosin or immunostained for α_{σ}β₃-integrin (LM-609, Chemicon International, Inc.) using the Vectastain® Elite ABC kit (Vector Laboratories), and developed with the Vector® VIP kit. Microscopic images were obtained using a Nikon E800 research microscope and digitized with a Nikon DXM1200 camera.

In the present studies, Vx-2 tumors were excised from the popliteal fossa to confirm their pathology and angiogenic features, which proved to be consistent with previous published images. In general the Vx-2 tumors were typically round and between 0.6 cm and 1.5 cm or less in their greatest dimension. The neovasculature was asymmetrically distributed within the peripheral tumor capsule with the greatest density appreciated along muscle tumor interfaces. Testis tissue, which presented a strong ^{99m}Tc α_{σ}β₃-nanoparticles contrast signal by SPECT-CT, was excised in two animals and examined for angiogenesis using anti-α_{σ}β₃-integrin antibody (LM 609). Prominent immunostaining for α_{σ}β₃-integrin clearly corroborated the in vivo nuclear signal observed, and also provided an independent, positive control site.

Statistical Analysis: Data were analyzed using general linear models, which included analysis of variance (located on the World Wide Web at r-project.org) and Student's t-test (GSL packages, located on the World Wide Web at gnu.org/software/gsl). Mean separations invoked the LSD method (p<0.05). Averaged data are presented as the mean±standard error of the mean unless otherwise stated.

### EXAMPLE 2

### Imaging with ¹¹¹In

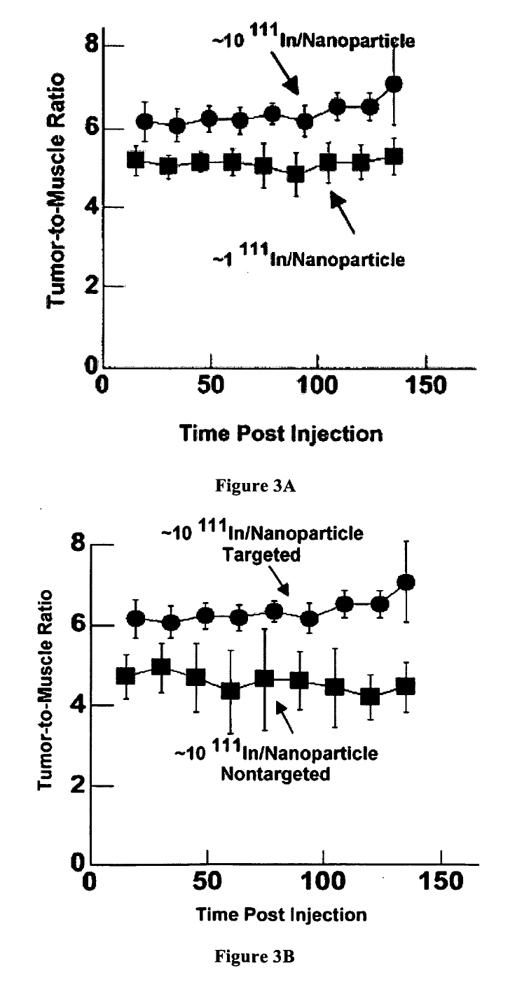

In protocols similar to those set forth in Example 1, the compositions of the invention were employed in the rabbit Vx-2 tumor model and similarly to Example 1, the tumor-to-muscle ratio of radioactivity compared. The results are shown for various dosages and combinations in FIGS. 3A-3B. FIG. 3A compares the effect of a 10-fold increase in dosage on the ratio and FIG. 3B compares targeted versus nontargeted nanoparticles at the same dosage level.

1. Use of an emulsion of nanoparticles targeted to α_{σ}β₃ which nanoparticles include a chelated radioisotope in a method to identify the location of neovasculature associated with a tumor as distinct from angiogenesis in normal tissue which method comprises
administering to a tumor-bearing subject an emulsion of said nanoparticles targeted to α_{σ}β₃ which nanoparticles include a chelated radioisotope and obtaining a high sensitivity low resolution image of neovasculature;
optionally followed by obtaining a high-resolution, low-sensitivity image of neovasculature said tumor.

2. The use of claim 1 wherein the high-sensitivity, low-resolution image of neovasculature in the tumor is compared to a similar image in muscle.

3. The use of claim 1 wherein the chelating agent is a compound of the formula (1) 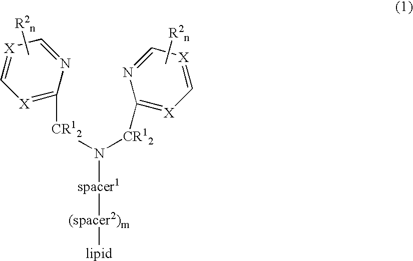
wherein;
each X is independently CR¹ or N;
each R¹ is independently H or lower alkyl;
each R² is independently halo, alkyl (1-6C), alkenyl (2-6C), or alkynyl (2-6C);
n is 0, 1 or 2;
spacer¹ is an alkylene or alkenylene chain of four or more carbons;
spacer², when present, couples spacer¹ to a lipid moiety and is a hydrophilic optionally substituted alkylene chain wherein one or more C may be replaced by N or O and wherein said chain may be substituted with one or more of OR, NR₂, ═O, COOR, CONR₂, OOCR, and/or NRCOR wherein each R is independently H or lower alkyl;
m is 0 or 1; and
lipid represents a fatty acid, a phospholipid, a sphingolipid or a steroid.

4. The use of claim 1 wherein the radioisotope is a ^{99m}Tc or ¹¹¹In.

5. A method to obtain an image of neovasculature associated with a tumor in a subject, which method comprises obtaining a high sensitivity, low resolution image of neovasculature in said subject in combination with obtaining a high resolution image of the neovasculature in the tumor in said subject.

6. The method of claim 5 wherein the high sensitivity, low resolution image is obtained using a chelated radioisotope and the chelating agent is a compound of the formula (1) 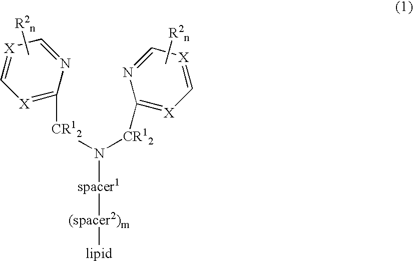
wherein;
each X is independently CR¹ or N;
each R¹ is independently H or lower alkyl;
each R² is independently halo, alkyl (1-6C), alkenyl (2-6C), or alkynyl (2-6C);
n is 0, 1 or 2;
spacer¹ is an alkylene or alkenylene chain of four or more carbons;
spacer², when present, couples spacer¹ to a lipid moiety and is a hydrophilic optionally substituted alkylene chain wherein one or more C may be replaced by N or O and wherein said chain may be substituted with one or more of OR, NR₂, ═O, COOR, CONR₂, OOCR, and/or NRCOR wherein each R is independently H or lower alkyl;
m is O or 1; and
lipid represents a fatty acid, a phospholipid, a sphingolipid or a steroid.

7. The method of claim 6 wherein the radioisotope is a ^{99m}Tc or ¹¹¹In.

8. A compound of the formula (1) 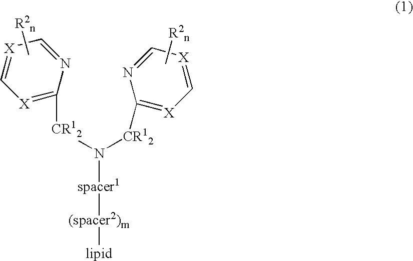
wherein;
each X is independently CR¹ or N;
each R¹ is independently H or lower alkyl;
each R² is independently halo, alkyl (1-6C), alkenyl (2-6C), or alkynyl (2-6C);
n is 0, 1 or 2;
spacer¹ is an alkylene or alkenylene chain of four or more carbons;
spacer², when present, couples spacer¹ to a lipid moiety and is a hydrophilic optionally substituted alkylene chain wherein one or more C may be replaced by N or O and wherein said chain may be substituted with one or more of OR, NR₂, ═O, COOR, CONR₂, OOCR, and/or NRCOR wherein each R is independently H or lower alkyl;
m is 0 or 1; and
lipid represents a fatty acid, a phospholipid, a sphingolipid or a steroid.

9. The compound of claim 8 which chelates a moiety comprising ^{99m}Tc or ¹¹¹In.

10. The compound of claim 8 wherein each R² is H.

11. The compound of claim 10 wherein each X represents CH.

12. The compound of claim 8 wherein spacer¹ is a residue of lysine.

13. The compound of claim 8 wherein spacer² is present and comprises polyethylene glycol.

14. The compound of claim 8 wherein spacer² comprises one or more amide linkages.

15. The compound of claim 8 wherein the lipid is phosphatidyl ethanolamine, phosphatidyl inositol, phosphatidyl glycine, phosphatidyl glycerol, or cholesterol.

16. The compound of claim 8 which is Bis-Py-Lys-Cap-PE or Bis-Py-Lys-PEG-cap-PE.

17. A composition comprising nanoparticles which nanoparticles have an outer lipid/surfactant layer, in which layer is embedded a multiplicity of molecules of formula (1) or Bis-Py-Lys-Cap-PE or Bis-Py-Lys-PEG-cap-PE.

18. The composition of claim 17 wherein the molecules of formula (1), Bis-Py-Lys-Cap-PE or Bis-Py-Lys-PEG-cap-PE chelate a moiety which comprises ^{99m}Tc or ¹¹¹In.

19. The composition of claim 17 wherein said nanoparticles are further coupled to a targeting ligand.

20. The composition of claim 18 wherein said nanoparticles are further coupled to a targeting ligand.

21. The composition of claim 19 wherein the targeting ligand comprises a peptidomimetic that binds specifically to α_{σ}β₃. or to fibrin.

22. The composition of claim 19 wherein the targeting ligand is coupled through a hydrophilic linker to a lipid moiety which is a fatty acid, a phospholipid, a sphingolipid or a steroid through a hydrophilic linker and wherein said lipid moiety is embedded in the lipid/surfactant layer of said nanoparticles.
